(19) United States
(12) Patent Application Publication
(10) Pub. No.: US 2012/0252862 A1
(43) Pub. Date: Oct. 4, 2012

(54) Dissolvable Film Strip Comprising Natural Components

(75) Inventors: Erdem Yesilada, Istanbul (TR); Gulengul Duman, Istanbul (TR)

(73) Assignee: YEDITEPE UNIVERSITES, Istanbul (TR)

(21) Appl. No.: 13/498,900

(22) PCT Filed: Sep. 14, 2010
(86) PCT No.: PCT/IB10/54121

§ 371 Date: Jun. 12, 2012

(30) Foreign Application Priority Data
Sep. 28, 2009 (TR) 2009/97338

(51) Int. Cl. A61K 31/05; A61K 31/353; A61K 31/4045; A61P 3/04; A61P 7/02; A61P 3/00; A61P 35/00; A61P 39/06; A61K 47/36; A61P 9/00

(52) U.S. Cl. 514/415; 514/777; 514/733; 514/456

(57) ABSTRACT

The present invention relates to film strips which comprise natural product formulations and can be absorbed from oral mucosa. The said film strip can be used with the aim of preventing and treating diseases in a short time, by being absorbed rapidly when it is applied from the oral mucosa at lower dosages than the dosage forms known.

### FIELD OF THE INVENTION

The present invention relates to film strips which comprise natural product formulations and can be absorbed from oral mucosa.

### BACKGROUND OF THE INVENTION

While the drugs are used in the treatment of diseases, some components provided in the structure thereof are taken into the body as well. Treatment of diseases can be possible by means of these active substances. For example, melatonin is the compound which has the most potent natural cell-protecting and anti-carcinogenic effect known. The melatonin compound shows its effect by enabling the free radicals to be removed. Melatonin is a hormone which is secreted at night in the human body. It came out in postmortem studies that the melatonin level is higher in people died at night. The average plasma melatonin level in adults is about 60 to 70 pg/ml. In addition, after it was claimed in 1978 that the breast cancer is a disease which emerges in the absence of melatonin, many clinical studies have been conducted in order to determine the melatonin secretion in cancer patients. In some human cell types, anti-tumor effects of pineal gland and melatonin were reported.

Another component having potent antioxidant effect is resveratrol. There is resveratrol in the structure of red wine which is a natural product. However there is no resveratrol in the composition of each red wine. Soil products used in making red wine may vary according to soil conditions, growing and climate conditions, even years. There are proanthocyanidins which give the red color and the astringent taste to red wine. These substances also have potent antioxidant properties.

Apart from these, green tea is a natural product which has potent antioxidant property. There are alkaloids such as caffeine in green tea, besides catechin together with EGCG (epigallocatechin gallate) and flavonoides. Use of green tea extract especially with the aim of weight-loss is becoming widespread recently, owing to its lipolytic property which has been determined scientifically in recent years.

Drugs used in treatment of diseases in pharmacological field can be prepared in different forms such as tablets, syrups, powders, etc. Some problems may be confronted during application of these drugs. Depending on the interaction of the drugs with food, their absorption in the stomach reduces and thus bioavailability of drugs in the body does not occur sufficiently. In order that bioavailability of drugs to be efficacious, nutrients or drugs should be given to the body in optimum amounts. For example, in order that the daily amount of resveratrol which is recommended to the user can be taken in natural ways, people need to consume 500-1000 ml red wine every day. In this case, serious health problems may occur such as liver damage depending on the alcohol taken. On the other hand, there is no resveratrol in each red wine. Therefore, it can not be known whether the wine consumed comprises resveratrol or not. Likewise, it can be possible to take EGCG into the body by consuming green tea in natural ways. However, it is needed to consume a great quantity of green tea. Consuming green tea in high quantity may lead to serious problems in liver and kidney functions. Consuming green tea in high quantity may also lead to constipation problem.

Another problem occurring during use of drugs is that, when the pills that are taken into the body by oral route are not swallowed with water they show ulceration effect. Depending on the formulation of the drug, the lesion effect may increase too.

However apart from these in drug property, there are pullulan structures in polysaccharide structure of which there is a polymer that does not have a pharmacological effect and which is composed of maltotriose molecules. They are on the market as film strips which can be absorbed from oral mucosa. For example, Listerine® is a product of pullulan structure which is used for oral hygiene and can be absorbed from oral mucosa. The said product is of daily use in order to give aroma to the mouth.

The Unites States patent document no. US2009004254 A1, an application in the state of the art, discloses active drugs in the form of film which are orally-dissolvable and edible. The said film takes form with a water-soluble polymer. The said drug comprises a binding agent which is dissolvable in the mouth of the patient and an active drug dispersed in this agent thus pharmacologically effective dose of the drug is taken into the body by dissolving in the mouth of the patient.

The Japanese patent document no. JP63093717 A, an application in the state of the art, discloses an agent comprising polymaltotriose and showing high adhesive property in oral mucosa. This agent can be used to fix the drugs to the mucosa in oral cavity. This adhesive agent can be applied by being added to the drug for treatment.

### SUMMARY OF THE INVENTION

The objective of the present invention is to realize a film strip which shows a potent antioxidant effect in a short time by being absorbed rapidly when it is applied from the oral mucosa at lower dosages than the dosage forms known and which comprises natural components.

Another objective of the present invention is to realize a film strip which shows a rapid effect without undergoing digestive processes in stomach and intestine and which comprises natural components.

Yet another objective of the invention is to realize a film strip which can be carried easily and which comprises natural components.

A further objective of the invention is to realize a film strip which can be taken by oral route without needing water and which comprises natural components.

A yet further objective of the invention is to realize a film strip which has a potent antioxidant effect and which comprises natural components.

A still further objective of the invention is to realize a natural film strip wherein no filler or additive is used.

An additional objective of the invention is to realize a film strip which is easy to produce.

### DETAILED DESCRIPTION OF THE INVENTION

In the preparation of the inventive orally-absorbable film strip, the mixture of natural component freeze dried by means of lyophilization and maltotriose polymers known as melatonin powder is mixed with distilled water in an amount between 0.001-0.5 g/ml. The obtained mixture is poured over a smooth flat and non-adhesive layer in the form of a thin film. The poured film is freeze dried in drying oven at relative humidity of 50% and room temperature overnight. The obtained film is cut thinly. Eventually, a natural component-maltotriose complex is obtained in the form of a thin film. The obtained film strip is elastic, adhesive and easily orally-dissolvable.

In an embodiment of the inventive orally-absorbable film strip, the natural component is red wine. Red wine is freeze dried by means of lyophilization and red wine powder is mixed with maltotriose polymer such that it will provide the amount of resveratrol that a person should take. Consequently, red wine-maltotriose complex is obtained. There is no resveratrol in all wines, however, it was determined that there is high proportion of resveratrol substance in wines made of Bo [symbol] azkere and Öküzgözü grapes which are produced in Turkey. Since there is no resveratrol in all wines, the wine to be lyophilized is chosen from Bo [symbol] azkere and Öküzgözü which are indigenous grape types. Absorption of resveratrol from the gastrointestinal system occurs in small intestine only in the ratio of 20%. It is known that the oral bioavailability of the resveratrol in powder form is low however its absorption from the mouth is known to increase by the changes made in the formulation (for example liposomal resveratrol). A higher level of absorption is provided when resveratrol inside red wine is applied orally in the form of a film strip, in comparison with the absorption from gastrointestinal system. Procyanidinins provided in the composition of red wine will increase absorption of resveratrol and contribute to effect owing to its antioxidant property as well. Furthermore, since the components in the film strip mix with blood directly upon being absorbed from the mouth they don't undergo gastrointestinal metabolism (first-pass metabolism). Thus the liver doesn't get tired and potential toxicity risks which may be caused by metabolites in the body can be eliminated.

The film strip in the structure of the red wine-maltotriose complex has the following effects: it dilutes blood (anti-platelet) in cardiovascular diseases, reduces the risk of heart attack depending on the antioxidant effect, increases good cholesterol (HDL) and prevents oxidation of bad cholesterol (LDL). In addition, it has been put forward by scientific studies that it has preventive and therapeutic effects in pathologies such as particularly smoking related cancers and prostate and anti-aging effect depending on the antioxidant effect thereof.

In another embodiment of the invention, green tea comprising EGCG (EpiGallo Catechin Gallate) agent is used. Accordingly, the green tea extract is freeze dried by means of lyophilization and the green tea powders are mixed with maltotriose polymer such that it will provide the amount of EGCG (EpiGallo Catechin Gallate) that a person should take. The product obtained as a thin film is in the structure of green tea-maltotriose complex.

It is reported that EGCG is safe to be applied up to 1600 mg as single oral dose.

Thin film strip comprising green tea is used with the aim of weight-loss owing to its lipolytic property. However, the use of caffeine in cardio-paths (because increases blood pressure) or those with gastric problems (because increases gastric acid) usually poses a problem. Therefore in an embodiment of the present invention, thin film strips are obtained in the structure of EGCG-maltotriose complex using directly pure EGCG which does not comprise caffeine.

In another embodiment of the inventive orally-absorbable film strip, melatonin is chosen as the natural component. Bioavailability of melatonin by oral route is 15%. Its absorption is fast since it is taken from mouth directly in the form of film and its mixture with blood and efficacy are high. Melatonin is a natural cell-protecting hormone which is secreted in the human body. The scientific studies made put forward that it has potent antioxidant property and anticancer effect. Thin film strip comprising melatonin is an appropriate formulation form for melatonin the use of which is gradually increasing in recent years and it is a product form which provides fast efficacious and easy use in order to alleviate jet-lag complaints in especially intercontinental journeys.

1.-13. (canceled)

14. A film strip comprising elastic, adhesive and orally-dissolvable natural components and obtained by carrying out the processes of
mixing a natural component freeze dried by means of lyophilization, with a maltotriose polymer and water,
pouring the mixture in the form of a thin film, and
drying the poured thin film.

15. The film strip according to claim 14, characterized in that the mixture ratio of natural component-maltotriose polymer is in the range of 0.001-0.5 g/ml.

16. The film strip according to claim 15, characterized in that the natural component lyophilized is resveratrol.

17. The film strip according to claim 16, characterized in that the natural component is red wine comprising resveratrol.

18. The film strip according to claim 15, characterized in that the natural component lyophilized is EGCG (EpiGallo Catechin Gallate).

19. The film strip according to claim 18, characterized in that the natural component is green tea comprising EGCG.

20. The film strip according to claim 15, characterized in that the natural component lyophilized is melatonin.

21. The film strip according to claim 17, characterized in that it is used to reduce the risk of heart attack, increase good cholesterol (HDL), prevent oxidation of bad cholesterol (LDL) and dilute the blood in cardiovascular diseases (antiplatelet).

22. The film strip according to claim 17, characterized in that it is used as a prophylactic and an adjuvant to treatment in a plurality of cancers including prostate disease.

23. The film strip according to claim 17, characterized in that it is used for anti-aging depending on an antioxidant effect thereof.

24. The film strip according to claim 18, characterized in that it is used in weight-loss programs with its lipolytic property.

25. The film strip according to claim 19, characterized in that it is used in weight-loss programs with its lipolytic property.

26. The film strip according to claim 20, characterized in that it is used as a natural cell-protector and an anti-carcinogen.

27. The film according to claim 20, characterized in that it is used to alleviate jet-lag effects in a body by enabling free radicals to be removed from the body.
